(19) United States
(12) Patent Application Publication
(10) Pub. No.: US 2012/0237935 A1
(43) Pub. Date: Sep. 20, 2012

(54) REAGENT KIT FOR QUANTITATIVELY DETECTING THE MUTATIONS OF EPIDERMAL GROWTH FACTOR RECEPTOR(EGFR)

(75) Inventors: Junpu XU, Beijing (CN); Zhao Chen, Beijing (CN); Jun Li, Beijing (CN)

(73) Assignee: Beijing ACCB Biotech Ltd., Beijing (CN)

(21) Appl. No.: 13/497,527

(22) PCT Filed: Sep. 20, 2010
(86) PCT No.: PCT/CN10/01445

§ 371 Date: Mar. 21, 2012

(30) Foreign Application Priority Data
Sep. 22, 2009 (CN) 200910176852.5

(51) Int. Cl. G01N 21/64; C12N 15/63

(52) U.S. Cl. 435/6.11; 435/320.1

(57) ABSTRACT

The present invention relates to a detection method and a detection kit for EGFR gene mutations, which relates to the therapeutic efficacy of molecular-targeted anti-cancer drugs. Particularly, the present invention relates to a fluorescent quantitative PCR method and kit for detecting mutations at hotspots of EGFR gene, together with the use thereof. The present invention detects the mutations at specific sites of EGFR gene, and can predict the therapeutic efficacy of EGFR tyrosine kinase inhibitors. Therefore, it can provide a guidance to individualize treatments for cancer patients.

### CROSS REFERENCE TO RELATED APPLICATIONS

This application claims the priority of Chinese Patent Application No. 200910176852.5, filed on Sep. 22, 2009, the disclosure of which is incorporated herein by reference.

### BACKGROUND OF THE INVENTION

Epidermal Growth Factor Receptor (EGFR), a multifunctional glycoprotein widely spread on cell membranes of human tissues, is a member of HER/ErbB family, and is related with tumor propagation, vascular-genesis, tumor metastasis and resistance to apoptosis.

Most of the tumor cells express EGFR and its natural ligand, which, after binding to each other, can cause self phosphorylation, and transfer signal into nucleus via series of reactions, thus influence tumor development and evolution by influencing the growth and apoptosis of tumor cells, and tumor vascular-genesis.

It has been reported that EGFR plays an important role in cancer initiation and development. Targeting drugs directed to EGFR, e.g. EGFR tyrosine kinase inhibitors (EGFR-TKIs) such as AVASTIN, Erlotinib and Irressa etc., can inhibit propagation, invasion, metastasis of tumor cells and vascular-genesis, and induce apoptosis of tumor cells, by blocking EGFR signal transduction in tumor cells.

However, clinical practice shows that only 8-18% of Non-small-cell lung carcinoma patient can benefit from EGFR TKIs such as Iressa etc. (Paez J G, et al, Science, 2004, 304: 1497-1500; Sequist L V, et al, Oncologist, 2007, 12(1):90-8). It is found that the therapy effect is significant for patients carrying EGFR gene mutation. The frequency of mutations is consistent to the population's sensitivity to Iressa: more women than men; more non-smokers than smokers; more adenocarcinomas than others, and more Eastern people than Western people. By analyzing the mutations of EGFR gene in Iressa sensitive patient tissues, it was found that most individuals had mutations in the EGFR gene tyrosine kinase region. These mutations mainly exist in Exon 18-21 (Chan S K, et al, Eur J Cancer, 2006, 42(1): 17-23). Therefore, by detecting the mutations in EGFR gene Exon 18-21, it is possible to predict the therapy effect of molecular-targeted drugs such as Iressa. Taking in consideration of the most common sites of EGFR mutations in Chinese population (Han Yu et al., Chinese Oncology Journal, 2007, 29(4):278-282; Guo Jian et al., Chinese Lung-Cancer Journal, 2007, 10(6): 504-507) and using real-time quantitative PCR, the present invention detects five kinds of mutations in EGFR Exon 18, 19 and 21, which are related to the therapy efficacy of molecular-targeted anti-tumor drugs EGFR-TKIs. The therapy efficacy of EGFR-TKIs will be predicted by using the method described in the present invention for detecting the EGFR gene mutations.

The detecting method of the present invention has the following advantages: easy manipulation, and easy standardization. Other methods, such as allele specific oligonucleotide probe hybridization method, are very much dependent on hybridization conditions, thus they need strict controlling of the experimental conditions. The restriction fragment length polymorphism method, on the other hand, needs a lot of human labor, and can not generate quantitative results. The method of the present invention has short experimental cycle, and can be completed with 2 hours. It doesn't need to verify the result by sequencing, whereas the direct sequencing and high resolution melting analysis need 4 days to 2 weeks. Sensitivity of the method of the present invention is high, which, after optimizing experimental conditions, can reach 1% for detecting mutations, whereas sensitivity of direct sequencing is 20-50%. Specificity of the method of the present invention is also high. Immunohistochemistry (IHC) method can easily get pseudo-positive and pseudo-negative results, and can not determine the position and types of point mutations. The unique advantage of the present invention is accurate quantification. By using absolute quantification method to analyze data, draw standard curve, and accurately determine the content of wild-type gene and mutant gene in the samples, one can obtain ratio of the mutant gene in the samples, which will be of help to clinical diagnosis and therapeutic selection. Further, the present invention is safe and non-toxic, other methods such as chemical breaking method of mismatch base need isotope and toxic chemical agents.

### SUMMARY OF THE INVENTION

The question that the present invention addresses is to provide a quantitative detection kit for EGFR gene mutations, which can quantitatively detect the following mutations: EGFR Exon 18 (SEQ ID NO:1; SEQ ID NO:2) position 2155 G substituted with A; Exon 19 (SEQ ID NO:1; SEQ ID NO:3) position 2235-2249 deletion; Exon 19 position 2236-2250 deletion; Exon 19 2254-2277 deletion; and Exon 21 (SEQ ID NO:1; SEQ ID NO:4) position 2573 T substituted with G.

To address the above question, the present invention provides quantitative detection kit containing a mixture comprising Taq enzyme, 10×Taq buffer, MgCl₂, dNTP mixture, PCR primers which can specifically amplify the sequences at EGFR gene mutation positions, and probes which can specifically identify wild-type sequences and mutant sequences, together with method of the detection.

(1) Separately design upstream and downstream primers around the mutation positions of Exon 18, 19 and 21 of EGFR gene; and design specific probes according to each mutant site. Said probes can specifically bind wild-type sequences or the mutant sequences to be detected at specific EGFR sites, so as to determine whether the tested mutations occur at said sites.

(2) To accurately and quantitatively determine the ratio of the EGFR mutations, standards were designed in the present invention.

(3) Use fluorescent quantitative PCR to detect the samples and standards.

(4) Obtain standard curves for quantitative detection from the detection results of the standards, and calculate the ratios of EGFR gene mutations to the total wild type EGFR gene in the samples to be tested.

Prior to said step (1) it further includes: extracting nucleic acid from the samples, purifying it and determining the content of it.

The probes for fluorescent quantitative PCR specifically bind the sequences at EGFR gene mutation sites under suitable PCR conditions. Preferably, said probes link a fluorescence reporter group at their 5′ end, and link a fluorescence quencher group at their 3′ end. Said fluorescence reporter group is selected from FAM, TET, HEX and ROX. Said fluorescence quencher group is selected from BHQ, TAMARA. Preferably, said reporter group is FAM, and said quencher group is BHQ. Preferably, the sequences of said probes are SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41 or SEQ ID NO: 42.

Said standards include at least one of plasmids, genome DNA or chemically synthesized sequences. Preferably, said standards include wide-type plasmids and/or mutant plasmids, wherein said wild-type plasmids include wild-type sequences of EGFR gene, and said mutant plasmids include mutant sequences of EGFR gene. More preferably, said standards are consisted of wild-type plasmids and/or mutant plasmids. Preferably, the wild-type sequences of EGFR gene included in said wild-type plasmids are SEQ ID NO:46, SEQ ID NO:48 or SEQ ID NO:52, and the mutant sequences of EGFR gene included in said mutant plasmids are SEQ ID NO:47, SEQ ID NO:49, SEQ ID NO:50, SEQ ID NO:51 or SEQ ID NO:53.

The tested samples include fresh tissue, paraffin embedded tissues, cell lines, blood, pleural effusion, peritoneal effusion, saliva, digestive juice, urine and feces.

Said primers are consisted of upstream primers and downstream primers. Preferably, said primers are SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 or SEQ ID NO: 16.

Said quantitative detection kit for EGFR gene mutations includes the agents selected from: the above-mentioned primers, probes and standards. Preferably, said kit further includes Taq enzyme, 10×Taq buffer, MgCl₂, and dNTP mixture. Preferably, the ratio of primers to probes is 2:1-10:1, and the ratio of forward primers to reverse primers is 1:3-3:1. Said standards include a mixture of said plasmids in a certain ratio, wherein the ratio of the content of wild-type plasmids to mutant plasmids is 0%-100%.

### BRIEF DESCRIPTION OF DRAWINGS

The accompanying drawings, which are incorporated in and form a part of this specification, illustrate embodiments of the technology and, together with the description.

FIG. 1 is a diagram showing the method for constructing the plasmid standards in Example 2.

FIG. 2 is a diagram showing the wild-type plasmid profile of Example 2, wherein the wild-type PCR product sequence is inserted into the carrier at the position marked with an arrow.

FIG. 3 is a diagram showing the result of sequencing the wild-type plasmid standard of Example 2, wherein Fig. A is the sequencing result of EGFR Exon 18 2155G (wild-type) plasmid, Fig. B is the sequencing result of plasmid in which position 2235-2249, 2236-2250 and 2254-2277 of EGFR Exon 19 being wild-type, Fig. C is the sequencing result of plasmid in which position 2573 of EGFR Exon 21 being T (wild-type).

FIG. 4 is a diagram showing the sequencing result of mutant plasmid standard of Example 2, wherein the mutant site is marked with an arrow. Fig. A shows the sequencing of EGFR Exon 18 mutant of position 2155 G→A; Fig. B shows the sequencing of EGFR Exon 19 mutant of position 2235-2249 deletion; Fig. C shows the sequencing of EGFR Exon 19 mutant of position 2236-2250 deletion; Fig. D shows the sequencing of EGFR Exon 19 mutant of position 2254-2277 deletion; and Fig. E shows the sequencing of EGFR Exon 21 mutant of position 2573 T→G.

FIG. 5 shows the amplification curve of the standard of Example 3, wherein Fig. A shows the amplification curve of the plasmid standard of EGFR Exon 18 2155G (wild-type); Fig. B shows the amplification curve of the plasmid standard of EGFR Exon 19 position 2235-2249, 2236-2250 and 2254-2277 being wild-type; Fig. C shows the amplification curve of the plasmid standard of EGFR Exon 21 position 2573 being T(wild-type); Fig. D shows the amplification curve of mutant plasmid standard of EGFR Exon 18 position 2155 being G→A; Fig. E shows the amplification curve of the mutant plasmid standard of EGFR Exon 19 position 2235-2249 deletion; Fig. F shows the amplification curve of mutant plasmid standard of EGFR Exon 19 position 2236-2250 deletion; Fig. G shows the amplification curve of mutant plasmid standard of EGFR Exon 19 position 2254-2277 deletion; Fig. H shows the amplification curve of mutant plasmid standard of EGFR Exon 19 position 2573 being T→G.

FIG. 6 shows a standard curve based on FIG. 4, wherein Fig. A is a standard curve of plasmid with EGFR Exon 18 2155G (wild-type); Fig. B is a standard curve of plasmid with EGFR Exon 19 position 2235-2249, 2236-2250 and 2254-2277 being wild-type; Fig. C is a standard curve of plasmid with EGFR Exon 19 position 2473 being T (wild-type); Fig. D is a standard curve of mutant plasmid with EGFR Exon 18 position 2155 being G→A; Fig. E is a standard curve of mutant plasmid with EGFR Exon 19 position 2235-2249 being deletion; Fig. F is a standard curve of plasmid with EGFR Exon 19 position 2236-2250 being deletion; Fig. G is a standard curve of plasmid with EGFR Exon 19 position 2254-2277 being deletion; Fig. H is a standard curve of plasmid with EGFR Exon 18 position 2573 being T→G.

FIG. 7 shows the amplification curve of fluorescent quantitative PCR of the wild-type (Fig. A) and T→G replacement (Fig. B) of EGFR Exon 21 position 2573 in a tissue sample; wild-type (Fig. C) and position 2235-2249 deletion (Fig. D) of EGFR Exon 19 in a tissue sample; EGFR Exon 19 position 2236-2250 deletion (Fig. E) in whole blood sample; EGFR position 2254-2277 deletion (Fig. F) in whole blood sample; and position 2155 wild-type (Fig. G) and G→A replacement (Fig. H) of EGFR Exon 18 in cell line sample.

FIG. 8 is a diagram of the quantitative method of the present invention.

### DETAILED DESCRIPTION OF PARTICULAR EMBODIMENTS

### Examples

The following examples are put forth so as to provide those of ordinary skill in the art with a complete disclosure and description of how to make and use the present invention, and are neither intended to limit the scope of what the inventors regard as their invention nor they intended to represent that the experiments below are all or the only experiments performed. The experiment conditions which are not indicated in the Examples, are generally conventional, such as those disclosed in “Molecular Cloning, A Laboratory Manual, 3^{rd} ed, (Sambrook J.)”, or those suggested by the manufacturer.

### Example 1

Extracting genome DNA from fresh human tumor tissues, paraffin embedded tissues, peripheral blood, pleural effusion, and human cell lines

The tumor cell lines we tested included cell lines of: non-small-cell carcinoma (NSCLC; A549, H460, H838 and H1703), breast cancer (MCF-7, BT474 and HuL100), malignant mesothelioma (H513, H2052, H290, MS-1 and H28), colon cancer (SW480), head and neck cancer (U87), cervical carcinoma (Hela), sarcoma (Mes-SA, Saos-2 and A204).

The fresh human tumor tissues, peripheral blood, paraffin embedded tissues we tested included: NSCLC, mesothelioma, colon cancer, malignant melanoma, renal carcinoma, esophagus cancer, thyroid carcinoma, malignant cancer and ovarian cancer.

Extraction of Sample DNA

DNA extracting kit from Qiagen Inc., Promega Inc., or Roche Inc. can be used to extract genome DNA from the samples. Content and purity of the extracted DNA can be determined by using Nanodrop ND 1000 (Gene Inc.) (ODD260/OD280 is about 1.8, OD260/OD230 is more than 2.0). For example, the sample DNA may be extracted using the DNA Extracting Kit (Promega Inc.) as follows:

1. DNA Extraction from Fresh Tissues

(1) cut a bean-sized tissue using scissors, put it into a mortar, cut it into pieces, and ground it into powder by adding liquor nitrogen.

(2) added 600 μl pre-cooled lysate into the mortar, blow it 6 times using 1 ml tip, sufficiently mixed the tissue powder and the lysate, transferred the mixture into a 1.5 ml EP tube, then turned it over 6 times, water bath under 65° C. for 20 minutes.

(3) added 3 μl RNase, turned over 6 times to mix homogenously, water bath under 37° C. for 20 minutes.

(4) cooled it to room temperature, added 200 μl protein precipitation agent, turned over 6 time to mix homogenously, placed it on ice for 5 minutes, 13000×g centrifuged 4 minute at room temperature.

(5) transferred the supernatant into a new EP tube pre-added with 600 μl isopropanol (room temperature), gently mixed 6 times, then 13,000×g centrifugated at room temperate for 1 minutes.

(6) discarded the supernatant, added 600 μl 70% ethanol (room temperature) into precipitate, 13,000×g centrifugated at room temperate for 1 minutes.

(7) removed ethanol, air drying for 15 minutes.

(8) added 40 μl DNA solution into the precipitate, incubated at 65° C. for 1 hour or 4° C. overnight.

2. DNA Extraction from Paraffin Embedded Tissues

(1) added 1 mg or less tissues into 1.5 ml centrifuge tube.

(2) added freshly prepared 100 μl incubation buffer/proteinase K solution, and incubated at 56° C. overnight based on the type of the samples.

(3) took out the incubated sample tube, added two times volume of lysate buffer.

(4) vortex oscillated the resin for 10 seconds until the resin fully suspended, added 7 μl fully suspended resin, vortex oscillated the resin for 3 seconds, then incubated at room temperature for 5 minutes.

(5) vortex oscillated the resin for 2 seconds, put the tube on a magnetic separation rack (MagneSphere®), immediately conducted magnetic separation.

(6) carefully removed all solution, without touching the resin on the tube wall.

(7) added 100 μl lysate buffer, got out the tube from the magnetic separation rack, vortex oscillate for 2 seconds.

(8) put the tube back to the magnetic separation rack, removed all the lysate.

(9) added 100 μl 1×washing fluid, got out the tube from the magnetic separation rack, vortex oscillated 2 seconds.

(10) put the tube back to the magnetic separation rack, removed all the lysate.

(11) repeated step (9) and (10) twice, totally washed three times, and removed all the liquid after the last wash.

(12) opened the lid, put the tube on the magnetic separation rack, air drying for 5 minutes.

(13) added 25 μl eluate.

(14) closed the lid, vortex oscillate for 2 seconds, incubated at 65° C. for 5 minutes.

(15) took out the incubated tube, vortex oscillated for 2 seconds, immediately put it on the magnetic separation rack.

(16) carefully transferred the DNA solution into a selected container.

3. DNA Extraction of Whole Blood

(1) obtained 300 μl anticoagulant whole blood, added 900 μl cell lysate, blow 6 times using 1 ml tip, so that the whole blood and the cell lysate were sufficiently mixed, placed it under room temperature for 10 minutes, blew with the tip three times.

(2) 13,000×g centrifugated under room temperature for 20 seconds, discarded the supernatant, shook violently, added 300 μl pre-cooling lysate, blew with 1 ml tip until the precipitate were totally dissolved.

(3) added 1.5 μl RNase, turned over 6 times to mix homogenously, water bath under 37° C. for 20 minutes.

(4) cooled to room temperature, added 100 μl protein precipitation agent, turned over 6 times to mix homogenously, placed it on ice for 5 minutes, 13,000×g centrifugated under room temperature for 4 minutes.

(5) transferred the supernatant to a new EP tube previously added 300 μl isopropanol (room temperature), gently mixed 6 times, centrifugated under room temperature for 1 minutes.

(6) discarded the supernatant, add 1 ml 70% ethanol (room temperature) into the precipitate, turned over 6 times to mix homogenously, 13,000×g centrifugated under room temperature for 1 minutes.

(7) removed ethanol, air drying for 15 minutes.

(8) added 40 μl DNA dissolving solution, stay at 65° C. for 1 hour or 4° C. overnight.

4. DNA Extraction of Pleural Effusion (1) obtained 5 ml pleural effusion, 2000 rpm centrifugated at room temperature for 10 minutes, removed the supernatant, added 1 ml cell lysate, turned over 6 times to mix homogenously, stayed under room temperature for 10 minutes.

(2) 13,000×g centrifugated under room temperature for 20 seconds, discarded the supernatant, shook violently, added 1 ml pre-cooling lysate, mixed until the precipitate totally dissolved.

(3) added 3 μl RNase, turned over 6 times to mix homogenously, water bath under 37° C. for 20 minutes.

(4) cooled to room temperature, added 200 μl protein precipitation agent, turned over 6 times to mix homogenously, placed it on ice for 5 minutes, 13,000×g centrifugated under room temperature for 4 minutes.

(5) transferred the supernatant to a new EP tube previously added 5 ml isopropanol (room temperature), gently mixed 6 times, 13,000×g centrifugated under room temperature for 1 minutes.

(6) discarded the supernatant, added 1 ml 70% ethanol (room temperature) into the precipitate, turned over 6 times to mix homogenously, 13,000×g centrifugated under room temperature for 1 minutes.

(7) sucked out ethanol, air drying for 15 minutes.

(8) added 40 μl DNA dissolving solution, stay at 65° C. for 1 hour or 4° C. overnight.

5. DNA Extraction from Cell Lines

(1) obtained at least 1×10⁶ cells, transferred them into a 1.5 ml EP tube, 13,000×g centrifugated at room temperature for 10 seconds. If the cells are adherent cells, they should be digested by trypsin before collecting them.

(2) discarded the supernatant, added 200 μl PBS to wash the cells, 13,000×g centrifugated under room temperature for 10 seconds, discarded the supernatant, shook violently until the precipitate was suspended.

(3) added 600 μl pre-cooling lysis solution, blew to mix homogenously with 1 ml tip until no visual cell blocks.

(4) added 3 μl RNase, turned over 6 times to mix homogenously, water bath under 37° C. for 20 minutes.

(5) cooled to room temperature, added 200 μl protein precipitation agent, turned over 6 times to mix homogenously, placed it on ice for 5 minutes, 13,000×g centrifugated under room temperature for 4 minutes.

(6) transferred the supernatant to a new EP tube previously added 600 μl isopropanol (room temperature), gently mixed 6 times, 13,000×g centrifugated under room temperature for 1 minutes.

(7) discarded the supernatant, added 600 μl 70% ethanol (room temperature) into the precipitate, turned over 6 times to mix homogenously, 13,000×g centrifugated under room temperature for 1 minutes.

(8) sucked out ethanol, air drying for 15 minutes.

(9) added 40 μl DNA dissolving solution, stayed at 65° C. for 1 hour or 4° C. overnight.

### Example 2

Preparation of the plasmid standards containing mutant and wild-type sequences

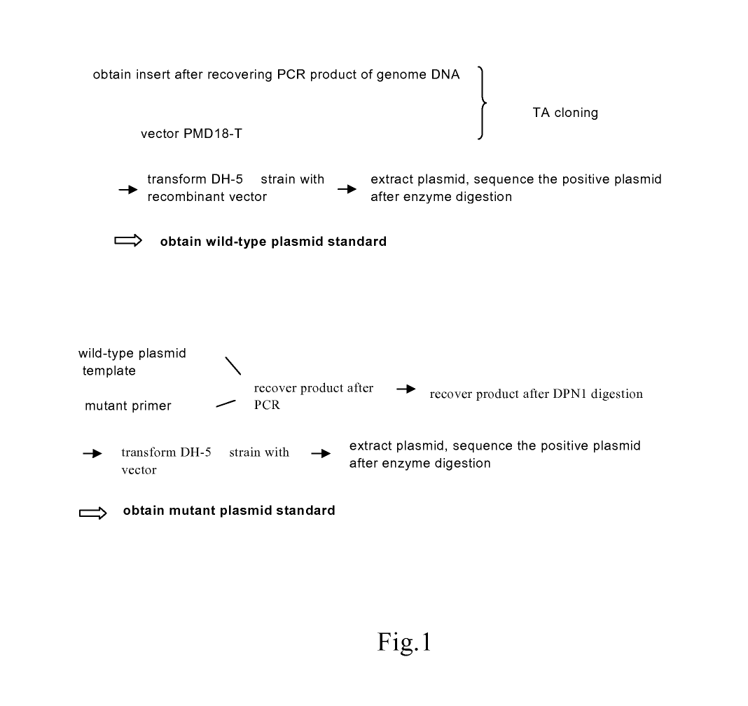

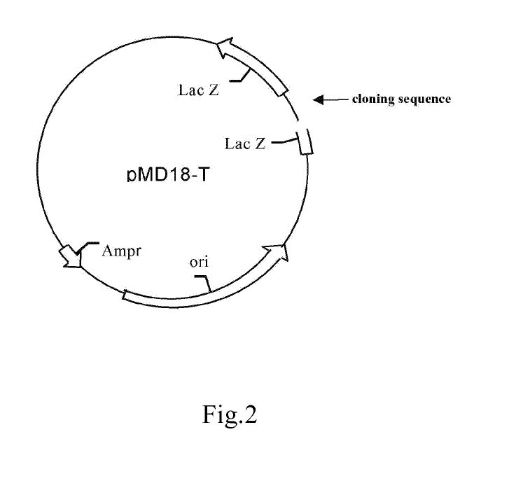

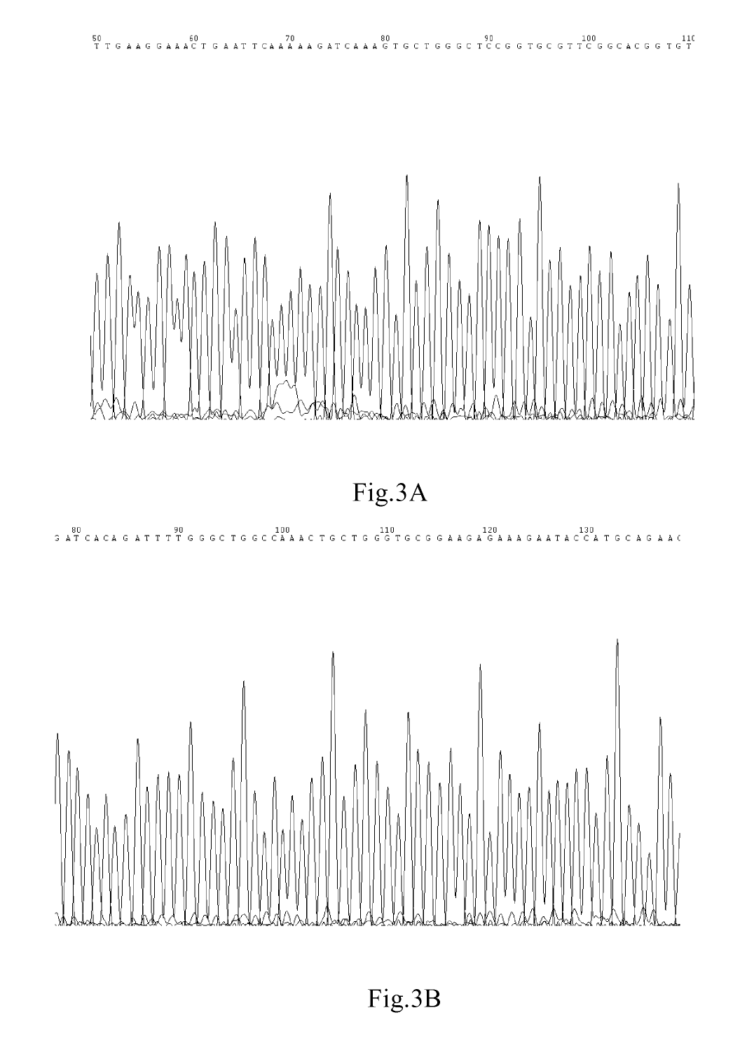

1. Construction of Wild-Type Plasmids (FIG. 1, FIG. 2)

1.1 Preparation of the Carrier

TA cloning carrier pMD18-T was purchased from TAKARA Inc.

1.2 Preparation of the Insert

The insert is prepared using PCR. The template of PCR is the sample genome DNA extracted in Step 1. The reaction system and amplification condition are shown in the following tables (Table 1, Table 2 and Table 3):

TABLE 1
PCR reaction system (50 μl)

| reagents | amount(μl/tube) |
|---|---|
| double-distilled water | 29.75 |
| 10 × buffer (free of Mg²⁺) | 5 |
| MgCl₂ (25 mM) | 7.5 |
| dNTP (10 mM) | 1.25 |
| upstream primer (25 μM) | 1.25 |
| downstream primer (25 μM) | 1.25 |
| Taq enzyme | 1 |
| DNA template | 3 |
| total volume | 50 |

For preparing the plasmid containing wild-type sequence at EGFR gene Exon 18 position 2155, it needs to add the primer sequences of E18-F-1 (SEQ ID NO:5) or E18-F-2 (SEQ ID NO:6) and E18-R-1 (SEQ ID NO:7) or E18-R-2 (SEQ ID NO:8) into the amplification system. For preparing the plasmid containing wild-type sequence of EGFR gene Exon 19 positions 2235-2249, 2236-2250 and 2254-2277, it needs to add the primer sequences of E19-F-1 (SEQ ID NO:9) or E19-F-2 (SEQ ID NO:10) and E19-R-1 (SEQ ID NO:11) or E19-R-2 (SEQ ID NO:12). For preparing the plasmid containing wild-type sequence at EGFR gene Exon 21 positions 2573, it needs to add the primer sequences of E21-F-1 (SEQ ID NO:13) or E21-F-2 (SEQ ID NO:14) and E21-R-1 (SEQ ID NO:15) or E21-R-2 (SEQ ID NO:16).

TABLE 2
PCR primers

| name | sequence | |
|---|---|---|
| E18-F-1 | GAGGATCTTGAAGGAAACTG | (SEQ ID NO: 5) |
| E18-F-2 | CCAGCTTGTGGAGCCTCTT | (SEQ ID NO: 6) |
| E18-R-1 | GCCAGGGACCTTACCTTAT | (SEQ ID NO: 7) |
| E18-R-2 | CTGTGCCAGGGACCTTACCTT | (SEQ ID NO: 8) |
| E19-F-1 | CCCAGAAGGTGAGAAAGTT | (SEQ ID NO: 9) |
| E19-F-2 | GGGACTCTGGATCCCAGAAG | (SEQ ID NO: 10) |
| E19-R-1 | CCTGAGGTTCAGAGCCAT | (SEQ ID NO: 11) |
| E19-R-2 | CCCACACAGCAAAGCAGAA | (SEQ ID NO: 12) |
| E21-F-1 | GCAGCCAGGAACGTACTGGT | (SEQ ID NO: 13) |
| E21-F-2 | CCCTCACAGCAGGGTCTTCT | (SEQ ID NO: 14) |
| E21-R-1 | GTGGGAAGGCAGCCTGGT | (SEQ ID NO: 15) |
| E21-R-2 | GTGGGAAGGCAGCCTGGT | (SEQ ID NO: 16) |

TABLE 3
PCR amplification condition

| steps | cycles | |
|---|---|---|
| Step 1 | 1 | 95° C., 1-5 minutes |
| Step 2 | 20-30 | 95° C., 10-15 seconds; |
| | | 55-65° C., 30-60 seconds |

1.3 After recovering the target fragment using QIAgen Gel Recover Kit, inserted said fragment into pMD18-T (purchased from TAKARA Inc.) by TA colonizing.

1.4 Amplified the constructed plasmid in E. coli DH5α strain, and harvested by extraction and purification (the methods are showed in Molecular Cloning, A Laboratory Manual, 3^{rd} ed. pages 96-99 and 103.

1.5 Identified the plasmid by double enzyme digestion of BamHI and HindIII.

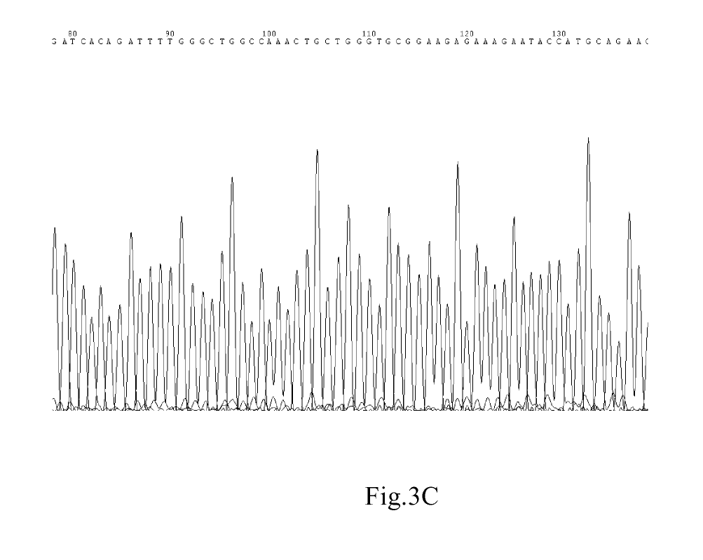

1.6 Sequenced the strains having positive result, and used the strains with correct sequence as the standard containing wild-type sequence (FIG. 3).

2. Construction of mutant plasmids: designed mutant primers of mutant sites, obtained the standards containing mutant sequences by DPN1 method.

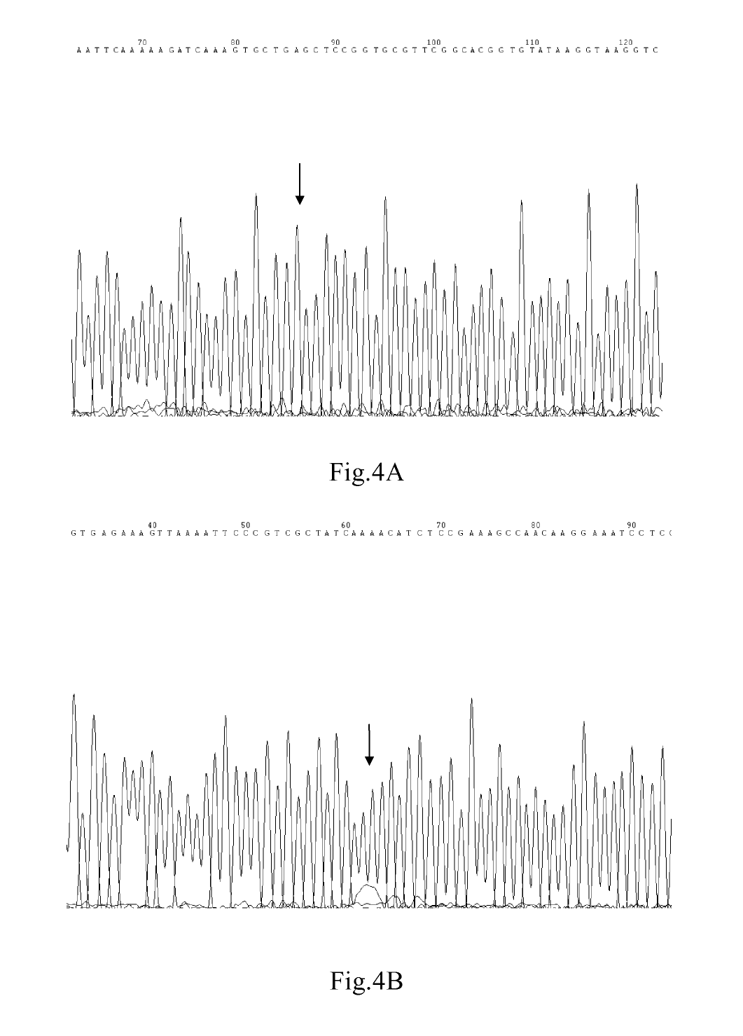

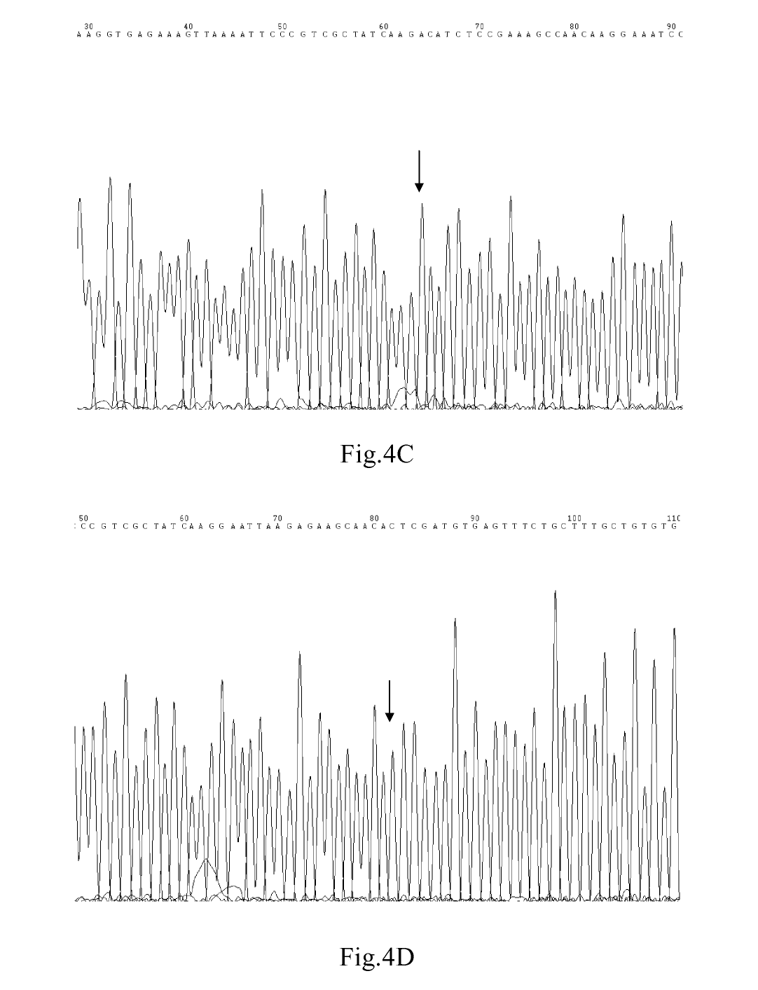

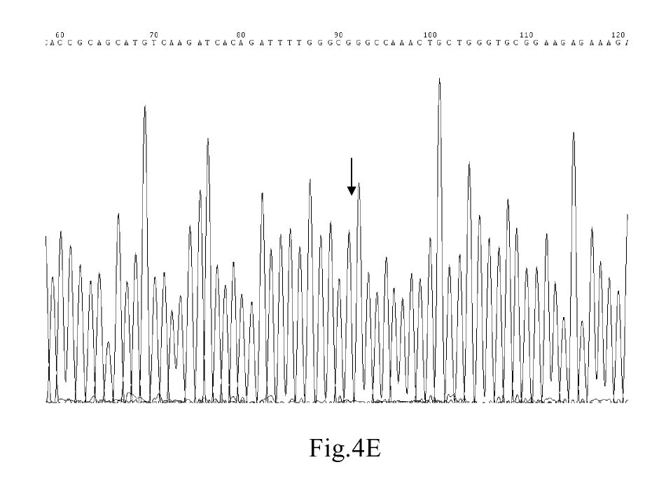

2.1 Designed the mutant primers (FIG. 4) of mutant sites based on the desired mutant sequences.

TABLE 4
mutant primers

| primers | | |
|---|---|---|
| name | sequences | |
| E18-M-F: | TGCTGAGCTCCGGTGCGTTCG | (SEQ ID NO: 17) |
| E18-M-R: | GGAGCTCAGCACTTTGATCTT | (SEQ ID NO: 18) |
| E19-1-F: | ATCAAAACATCTCCGAAAGCC | (SEQ ID NO: 19) |
| E19-1-R: | ATGTTTTGATAGCGACGGGAA | (SEQ ID NO: 20) |
| E19-2-F: | TCAAGACATCTCCGAAAGCCA | (SEQ ID NO: 21) |
| E19-2-R: | GATGTCTTGATAGCGACGGGA | (SEQ ID NO: 22) |
| E19-3-F: | CAACACTCGATGTGAGTTTCT | (SEQ ID NO: 23) |
| E19-3-R: | TCGAGTGTTGCTTCTCTTAAT | (SEQ ID NO: 24) |
| E21-M-F: | TGGGCGGGCCAAACTGCTGGG | (SEQ ID NO: 25) |
| E21-M-R: | TGGCCCGCCCAAAATCTGTGA | (SEQ ID NO: 26) |

2.2 Used 5 ng wild-type plasmid as template, and used mutant primers and Pfu enzyme to mutate the target sites. The amplification system and condition are shown in Table 1, Table 4 and Table 3.

During the preparation of the plasmid containing the mutant sequence with EGFR gene Exon 18 position 2155 G→A, it needs to add E18-M-F (SEQ ID NO:17) and E18-M-R (SEQ ID NO:18) primers into the amplification system. During the preparation of the plasmid containing the mutant sequence with EGFR gene Exon 19 position 2235-2249 deletion, it needs to add E19-1-F (SEQ ID NO:19) and E19-1-R (SEQ ID NO:20) primers into the amplification system. During the preparation of the plasmid containing the mutant sequence with EGFR gene Exon 19 position 2236-2250 deletion, it needs to add E19-2-F (SEQ ID NO:21) and E19-2-R (SEQ ID NO:22) primers into the amplification system. During the preparation of the plasmid containing the mutant sequence with EGFR gene Exon 19 position 2254-2277 deletion, it needs to add E19-3-F (SEQ ID NO:23) and E19-3-R (SEQ ID NO:24) primers into the amplification system. During the preparation of the plasmid containing the mutant sequence with EGFR gene Exon 21 position 2573 T→G, it needs to add E21-M-F (SEQ ID NO:25) and E21-M-R primer (SEQ ID NO:26) primers into the amplification system.

2.3 treated the product obtained in step 2.2 with DPN1 enzyme, recovered the product after incubating at 37° C. for 1 hour, amplified in E. coli DH5α strain, and harvested by extraction and purification.

2.4 Identified the plasmid by double enzyme digestion of BamHI and HindIII.

2.5 Sequenced the strains having positive result, and used the strains with correct sequence as the standard containing mutant sequence (FIG. 4).

### Example 3

Detection of EGFR mutations from genome DNA of human cell lines, human fresh tumor tissues, peripheral blood, and paraffin embedded tissues, using lung cancer and cervical carcinoma as examples.

1. The templates for fluorescent quantitative PCR were the genome DNA of lung cancer and cervical carcinoma samples extracted in Example 1, and the standards prepared in Example 2. Double-distilled water was served as negative control. For drawing the standard curves, the standards were diluted as 1 ng/μl. 0.5 ng/μl, 0.25 ng/μl, 0.125 ng/μl, 0.0625 ng/μl, 0.03125 ng/μl.

2. The reaction system and condition are shown in Table 2, Table 5, Table 6 and Table 7, wherein the fluorescent emission group bound to the probe is selected from FAM, TET, HEX or ROX, the quench group is selected from BHQ or TAMARA.

TABLE 5
Reaction system for fluorescent quantitative PCR (20 μl/tube)

| reagent | amount (μl/tube) |
|---|---|
| double-distilled water | 9.9 |
| 10 × buffer (free of Mg²⁺) | 2 |
| MgCl₂ (25 mM) | 3 |
| dNTP (10 mM) | 0.5 |
| upstream primer (25 μM) | 0.5 |
| downstream primer (25 μM) | 0.5 |
| fluorescent probe (25 μM) | 0.2 |
| Taq enzyme | 0.4 |
| DNA template | 3 |
| total volume | 20 |

For detecting the G→A mutation at EGFR gene Exon 18 position 2155, it needs to prepare two systems, i.e. separately add E18-F-1 (SEQ ID NO:5) and E18-F-2 (SEQ ID NO:6) and E18-R-1 (SEQ ID NO:7) or E18-R-2 (SEQ ID NO:8) primers, E18W-1 (SEQ ID NO:27) or E18W-2 (SEQ ID NO:28) probe, E18-F-1 (SEQ ID NO:5) or E18-F-2 (SEQ ID NO:6) and E18-R-1 (SEQ ID NO:7) or E18-R-2 (SEQ ID NO:8) primers, E18M-1 (SEQ ID NO:29) or E18W-2 (SEQ ID NO:30) probe.

For detecting the Exon 19 deletions (positions 2235-2249, 2236-2250 and 2254-2277), it needs to prepare 4 systems, i.e. separately add E19-F-1 (SEQ ID NO:9) or E19-F-2 (SEQ ID NO:10) and E19-R-1 (SEQ ID NO:11) or E19-R-2 (SEQ ID NO:12) primers, E19W-1 (SEQ ID NO:31) or E19W-2 (SEQ ID NO:32) probe; E19-F-1 (SEQ ID NO:9) or E19-F-2 (SEQ ID NO:10) and E19-R-1 (SEQ ID NO:11) or E19-R-2 (SEQ ID NO:12) primers, E19M1-1 (SEQ ID NO:33) or E19M1-2 (SEQ ID NO:34) probe, E19-F-1 (SEQ ID NO:9) or E19-F-2 (SEQ ID NO:10) and E19-R-1 (SEQ ID NO:11) or E19-R-2 (SEQ ID NO:12) primers, E19M2-1 probe (SEQ ID NO:35) or E19M2-2 (SEQ ID NO:36); E19-F-1 (SEQ ID NO:9) or E19-F-2 (SEQ ID NO:10) or E19-R-1 (SEQ ID NO:11) or E19-R-2 (SEQ ID NO:12) primers, E19M3-1 (SEQ ID NO:37) or E19M3-2 (SEQ ID NO:38) probe.

For detecting the T→G mutation at Exon 21 position 2573, it needs to prepare two systems, i.e., separately add E21-F-1 (SEQ ID NO:13) or E21-F-2 (SEQ ID NO:14) and E21-R-1 (SEQ ID NO:15) or E21-R-2 (SEQ ID NO:16) primers, E21W-1 (SEQ ID NO:39) or E21W-2 (SEQ ID NO:40) probe, 21-F-1 (SEQ ID NO:13) or E21-F-2 (SEQ ID NO:14) and E21-R-1 (SEQ ID NO:15) or E21-R-2 (SEQ ID NO:16) primers, E21M-1 (SEQ ID NO:41) or E21M-2 (SEQ ID NO:42) probe.

TABLE 6
Probes

| names | sequences | |
|---|---|---|
| E18W-1 | GGCTCCGGTGCGTTCGGC | (SEQ ID NO: 27) |
| E18W-2 | CGGAGCCCAGCACTTTGATCT | (SEQ ID NO: 28) |
| E18M-1 | TGCTGAGCTCCGGTGCGTT | (SEQ ID NO: 29) |
| El8M-2 | CGGAGCTCAGCACTTTGATCTT | (SEQ ID NO: 30) |
| El9W-1 | TCAAGGAATTAAGAGAAGCAACATC | (SEQ ID NO: 31) |
| E19W-2 | CGGAGATGTTGCTTCTCTTAATTCCT | (SEQ ID NO: 32) |
| El9M1-1 | CCCGTCGCTATCAAAACATCT | (SEQ ID NO: 33) |
| El9M1-2 | AGATGTTTTGATAGCGACGGG | (SEQ ID NO: 34) |
| El9M2-1 | CCCGTCGCTATCAAGACATCTC | (SEQ ID NO: 35) |
| El9M2-2 | GAGATGTCTTGATAGCGACGGG | (SEQ ID NO: 36) |
| E19M3-1 | AATTAAGAGAAGCAACACTCGAT | (SEQ ID NO: 37) |
| E19M3-1 | ATCGAGTGTTGCTTCTCTTAATT | (SEQ ID NO: 38) |
| E21W-1 | TGGCCAGCCCAAAATCTGTG | (SEQ ID NO: 39) |
| E21W-1 | AAGATCACAGATTTTGGGCTGGC | (SEQ ID NO: 40) |
| E21M-1 | TGGCCCGCCCAAAATCTGT | (SEQ ID NO: 41) |
| E21M-1 | GATCACAGATTTTGGGCGGGC | (SEQ ID NO: 42) |

TABLE 7
Amplification condition

| steps | cycles | |
|---|---|---|
| step 1 | 1 | 95° C., 1-5 minutes |
| step 2 | 30-45 | 95° C., 10-15 seconds; 55-65° C. |
| | | (collect fluorescent), 30-60 seconds |

3. Drawing the Standard Curve

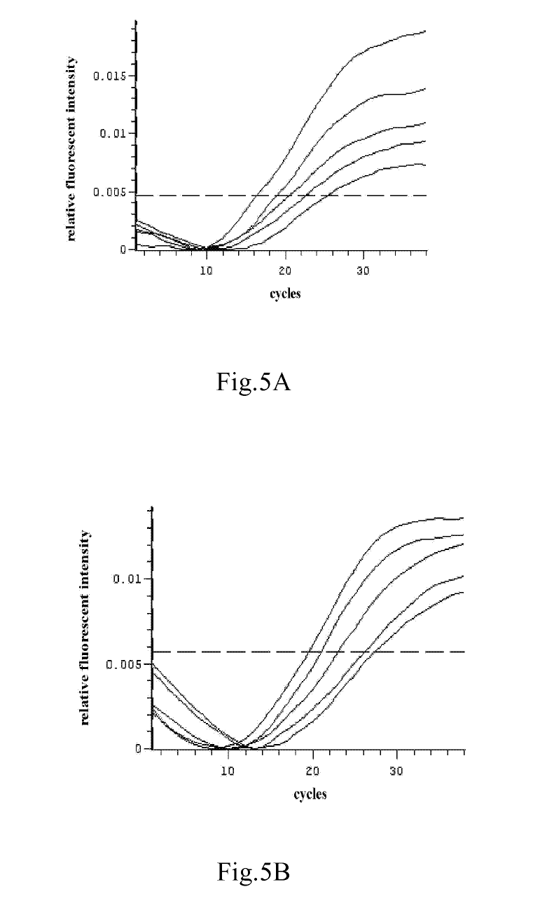

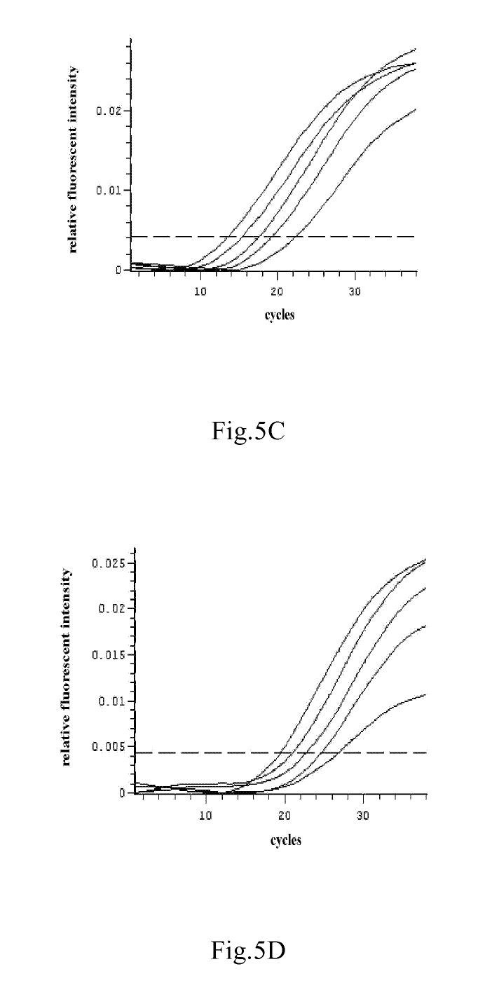

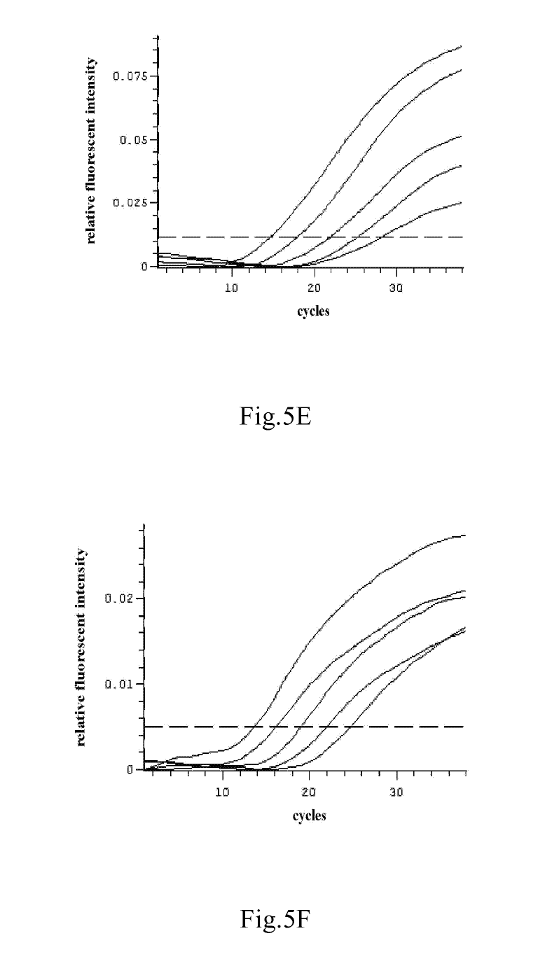

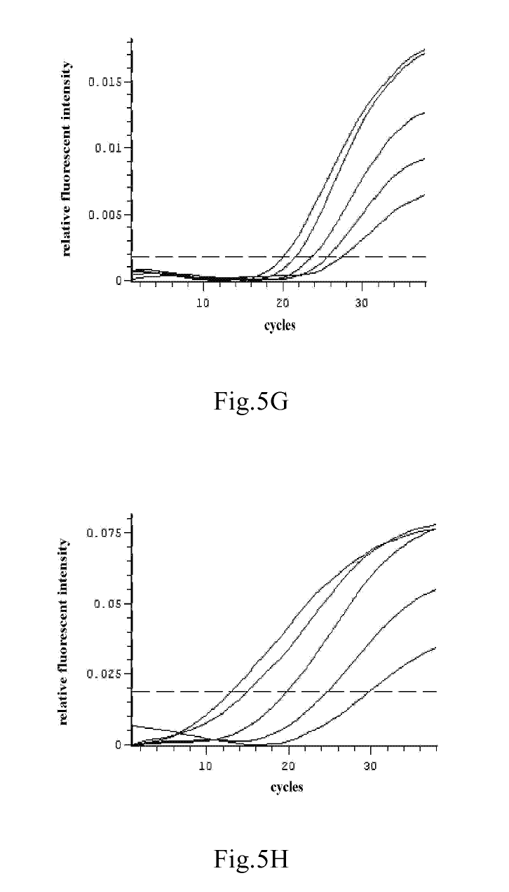

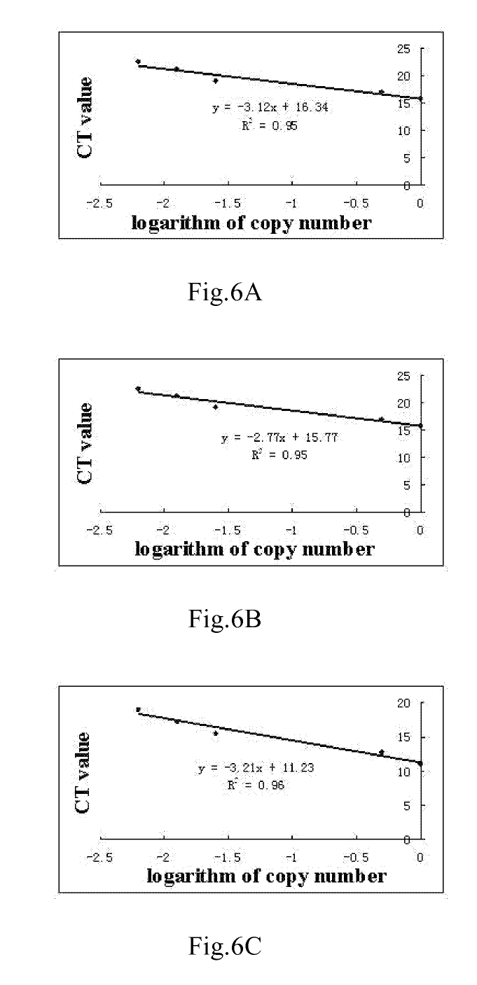

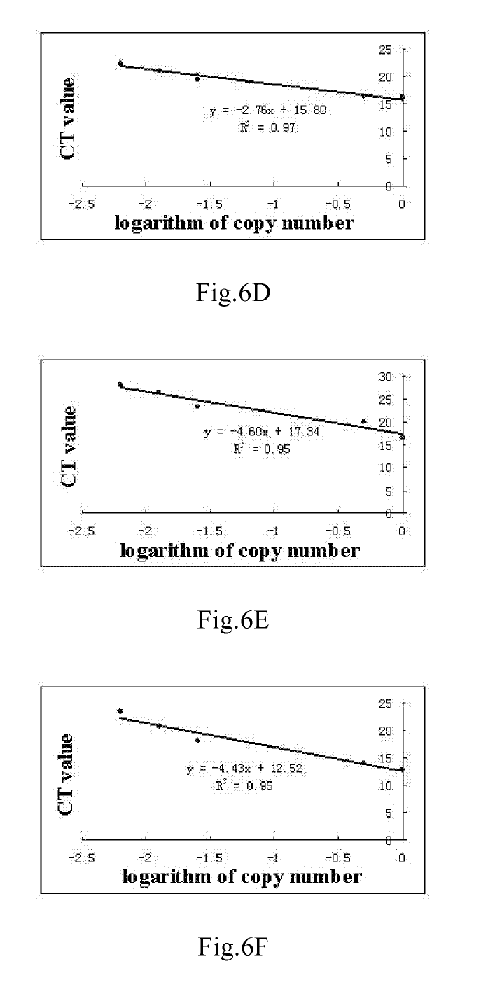

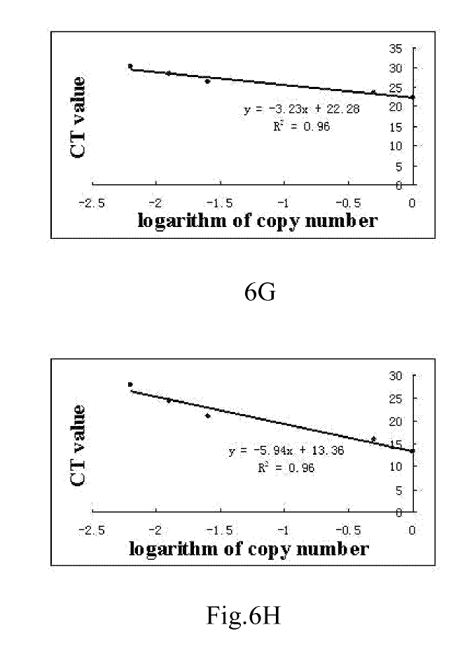

The standard curve was drawn based on the CT values obtained from the standard in Step 3. FIG. 5 shows the amplification curve of plasmid standard, in which the five rising curves represent, from left to right, the amplification curve of the plasmid standard with the dilute ratio of 0.5 ng/μl, 0.25 ng/μl, 0.125 ng/μl, 0.0625 ng/μl and 0.03125 ng/μl respectively. The horizontal axis represents cycle number, and the vertical axis represents fluorescent detection value. Accordingly, it is possible to draw the standard curve for calculation (FIG. 6). In FIG. 6, the horizontal axis represents the logarithm of copy number of the template, the vertical axis represents CT value, wherein the copy number of template=mass/(molecular weight)×6.02×10²³, the molecular weight of plasmid≈the number of bases×324.5, or is calculated using the software DNAMAN. In the present experiment, the plasmid is consisted of pMD18-T carrier and an insert. Because the lengths of the insert are almost identical, the biggest difference only lies in twenties bases, which can be ignored with respect to the length of 2692 bp for PMD18-T carrier. Therefore, the ratio of copies of wild-type to mutant plasmid standard≈the ratio of weight. The molecular weight of the plasmid of the present experiment≈890˜900 Kda, so the copy number of 1 ng plasmid is about 6.69×10⁸˜6.76×10⁸.

4. Calculation of the Ratio of Specific EGFR Mutation in a Sample

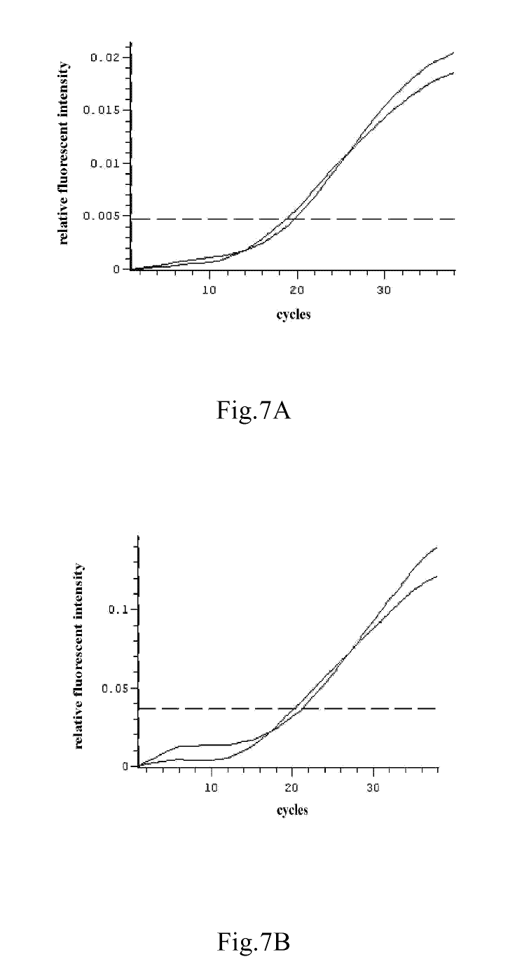

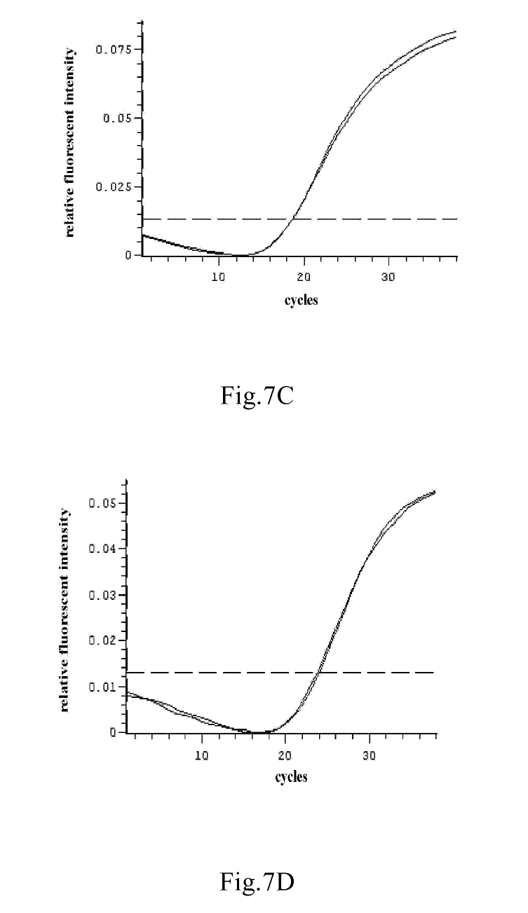

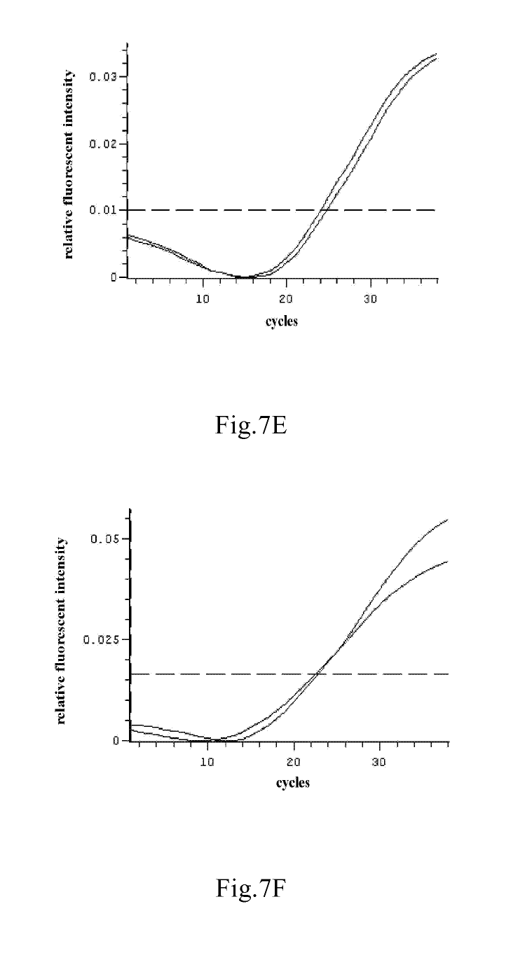

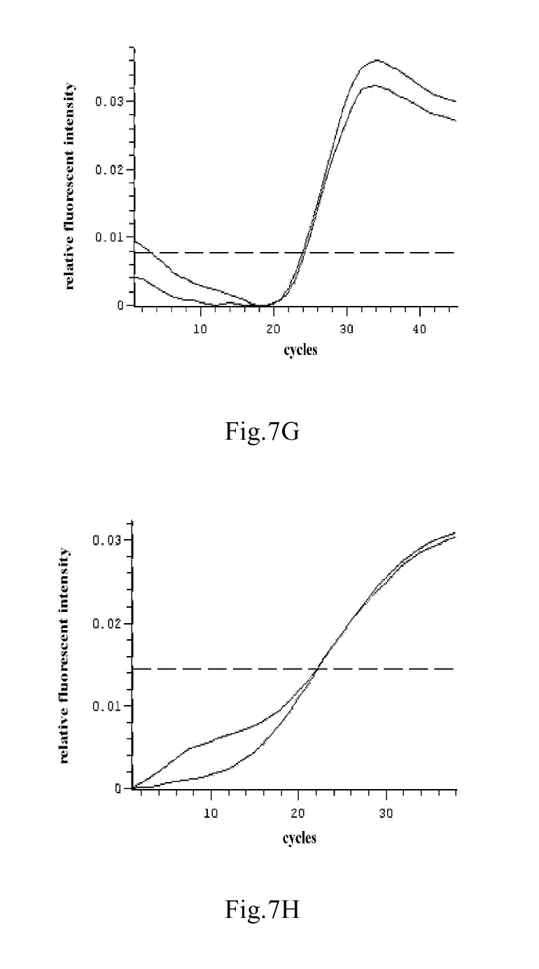

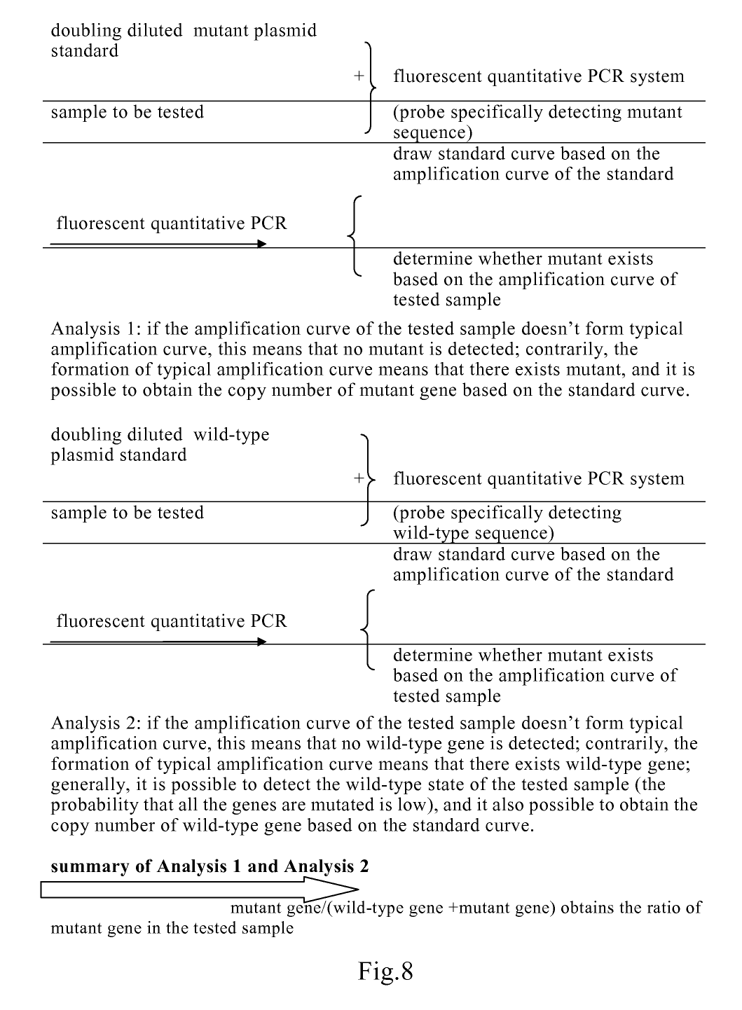

According to the standard curve, the copy numbers of wild-type and mutant genome DNA were calculated from the CT values of the sample. Then we obtained the ratio of mutant EGFR DNA to total EGFR DNA (wild-type plus all mutants at said site). As shown in FIG. 7, the wild-type CT value of EGFR Exon 21 of a tissue sample was 19.15, whereas the value of T→G mutation at position 2573 was 20.74. According to each standard curve formula (FIG. 6), we could calculate the copy numbers for them. We then obtained the ratio of the content of mutant to wild-type which was 89:100, and we estimated that about 47% EGFR gene in the tissue sample had T→G mutation at position 2573.

5. Result of Detection

In this Example, we detected the EGFR gene mutation in 48 cases of tissues, whole blood and cell line samples of lung cancer and cervical carcinoma, and found that 13 cases had mutations, and the concrete number can be seen in Table 8. The mutation ratios, i.e. the ratio of mutant gene to non-mutant gene in those samples, can be seen in Table 9.

TABLE 8
EGFR mutant cases

| mutation type | case number |
|---|---|
| Exon 18 2155 G→A | 1 |
| Exon 19 2235→2249Del | 3 |
| Exon 19 2236→2250Del | 1 |
| Exon 19 2254→2277Del | 2 |
| Exon 21 L858R | 6 |
| total | 13 |

TABLE 9
EGFR mutation ratio

| type of mutant | | |
|---|---|---|
| samples | mutation type | mutation ratio |
| H1975 cell line | Exon 21 L858R | 25% |
| cervical | Exon 19 2235→2249Del | 30% |
| carcinoma | Exon 19 2254→2277Del | 15% |
| tissue | | |
| lung cancer | Exon 18 2155 G→A | 32% |
| tissue | Exon 19 2235→2249Del | 18% |
| | Exon 19 2236→2250Del | 28% |
| | Exon 19 2254→2277Del | 15% |
| | Exon 21 L858R | 10%, 12%, 15%, |
| | | 15%, 47% |
| cervical | Exon 19 2235→2249Del | 25% |
| carcinoma | | |
| whole blood | | |

### Example 4

Detection of EGFR mutations from mRNA of human cell lines, human fresh tumor tissues, peripheral blood, and paraffin embedded tissues

1. Extracted Total RNA from Samples

Use Total RNA Extraction Kit (Invotrogen Inc., OMEGA Inc.) to extract RNA from the samples, and use Nanodrop ND1000 spectrophotometer (Gene Inc.) to detect the concentration and purification of the RNA (OD260/280 is between 1.8-2.0, OD260/230>2.0).

2. Use M-MLV reverse-transcripatase to conduct reverse transcription. The steps and reaction system are shown in Table 10:

TABLE 10
reverse transcription system (10 μl) and reaction steps

| reagent | amount (μl/tube) |
|---|---|
| RNA template | 5.5 |
| OligodT | 0.4 |

70° C. denatured 5 min, ice incubation 2-5 min, add the following agent

| | |
|---|---|
| 5 × buffer | 2 |
| dNTP(5 mM) | 1 |
| DEPC water | 0.35 |
| RNasin(40 U) | 0.25 |
| MLV | 0.5 |
| RT-enzyme | |
| Total | 10 |

37-42° C. 60-90 min, 70° C. 5 min_{∘}

3. The preparation method of the standards and the steps of real-time fluorescent PCR detection were similar to those in Example 1. The primers for preparing wild-type plasmid standards and the probes for fluorescent quantitative PCR detection were the same with those in Example 1, except that the primers for preparing wild-type plasmid standards and fluorescent quantitative PCR detection were different (Table 11). The method for drawing standard curve and calculating the content of specific mutations of EGFR gene were the same with those in Example 1.

TABLE 11
The primers for preparing wild-type plasmid
standards and for fluorescent quantitative PCR
detection of the samples when using cDNA as
template

| name | sequence | |
|---|---|---|
| E18-F-1 | GAGGATCTTGAAGGAAACTG | (SEQ ID NO: 5) |
| E18-R-3 | TGGGATCCAGAGTCCCTT | (SEQ ID NO: 43) |
| E19-F-1 | CCCAGAAGGTGAGAAAGTT | (SEQ ID NO: 9) |
| E19-R-3 | GTCTTTGTGTTCCCGGACAT | (SEQ ID NO: 44) |
| E21-F-1 | GCAGCCAGGAACGTACTGGT | (SEQ ID NO: 13) |
| E21-R-3 | GCCTCCTTCTGCATGGTATT | (SEQ ID NO: 45) |

Choose the primers for preparing wild-type plasmid standards:

For preparing the plasmid containing EGFR gene Exon 18 wild-type sequence at position 2155, it needs to add E18-F-1 (SEQ ID NO:5) and E18-R-3 (SEQ ID NO:43) primers. For preparing the plasmid containing EGFR gene Exon 19 wild-type sequences at positions 2235-2249, 2236-2250 and 2254-2277, it needs to add E19-F-1 (SEQ ID NO:9) and E19-R-3 (SEQ ID NO:44). For preparing the plasmid containing EGFR gene Exon 21 wild-type sequences at position 2573, it needs to add E21-F-1 (SEQ ID NO:13) and E21-R-2 (SEQ ID NO:45) primers.

Choose the primers and probes for fluorescent quantitative PCR detection:

For detecting the G→A mutation at EGFR gene Exon 18 position 2155, it needs to prepare two systems, i.e. separately add E18-F-1 (SEQ ID NO:5) and E18-R-3 (SEQ ID NO:43) primers, E18W-1 (SEQ ID NO:27) or E18W-2 (SEQ ID NO:28) probe, E18-F-1 (SEQ ID NO:5) and E18-R-3 (SEQ ID NO:43) primers, E18M-1 (SEQ ID NO:29) or E18W-2 (SEQ ID NO:30) probe.

For detecting the deletions in Exon 19 at positions of 2235-2249, 2236-2250, and 2254-2277, it needs to prepare 4 systems, i.e., separately add E19-F-1 (SEQ ID NO:9) and E19-R-3 (SEQ ID NO:44) primers, E19W-1 (SEQ ID NO:31) or E19W-2 (SEQ ID NO:32) probe, E19-F-1 (SEQ ID NO:9) and E19-R-3 (SEQ ID NO:44) primers, E19M1-1 (SEQ ID NO:33) or E19M1-2 (SEQ ID NO:34) probe, E19-F-1 (SEQ ID NO:9) and E19-R-3 (SEQ ID NO:44) primers, E19M2-1 probe (SEQ ID NO:35) or E19M2-2 (SEQ ID NO:36), E19-F-1 (SEQ ID NO:9) and E19-R-3 (SEQ ID NO:44) primers, E19M3-1 (SEQ ID NO:37) or E19M3-2 (SEQ ID NO:38) probe.

For detecting the T→G mutation at Exon 21 position 2573, it needs to prepare two systems, i.e. separately add E21-F-1 (SEQ ID NO:13) and E21-R-3 (SEQ ID NO:45) primers, E21W-1 (SEQ ID NO:39) or E21W-2 (SEQ ID NO:40) probe; E21-F-1 (SEQ ID NO:13) and E21-R-3 (SEQ ID NO:45) primers, E21M-1 (SEQ ID NO:41) or E21M-2 (SEQ ID NO:42) probe.

1-14. (canceled)

15. An assay kit for quantitatively detecting an EGFR gene mutation, comprising:
(1) a PCR primer which binds with nucleotides within a sequence under a suitable PCR condition, said sequence having 200 bases and comprising a mutation site of the EGFR gene;
(2) a probe for fluorescent quantitative PCR, said probe specifically binding to the base sequence at said mutation site of the EGFR gene under a suitable PCR condition; and
(3) a standard comprising a wild-type plasmid, mutant plasmid, or both a wild-type plasmid and a mutant plasmid, said wild-type plasmid comprising a wild-type EGFR sequence, and said mutant plasmid comprising a mutant EGFR sequence.

16. The kit according to claim 15, wherein said primer comprises a mixture of a upstream primer and a downstream primer.

17. The kit according to claim 15, wherein said primer is selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

18. The kit according to claim 15, wherein said probe is selected from the group consisting of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41 and SEQ ID NO: 42.

19. The kit according to claim 15, wherein said probe is linked to a fluorescent emitting group at its 5′ end, and is linked to a fluorescent quench group at its 3′ end.

20. The kit according to claim 19, wherein said fluorescent emitting group is FAM, TET, HEX, or ROX; and said fluorescent quench group is BHQ or TAMARA.

21. The kit according to claim 15, wherein the ratio of said primer to probe is 2:1-10:1, and said primer comprises a forward primer and reverse primer in a ratio of 1:3-3:1.

22. The kit according to claim 15, wherein said wild-type EGFR sequence in said wild-type plasmid is SEQ ID NO:46, SEQ ID NO:48 or SEQ ID NO:52.

23. The kit according to claim 15, wherein said mutant plasmid comprises a mutation which is in a form of base G or A at position 2155 in EGFR Exon 18; a deletion from position 2235 to position 2249, from position 2236 to 2250, or from position 2254 to 2277 in Exon 19; or base T or G at position 2573 in Exon 21.

24. The kit according to claim 15, wherein said mutant EGFR sequence in said mutant plasmid is SEQ ID NO:47, SEQ ID NO:49, SEQ ID NO:50, SEQ ID NO:51 or SEQ ID NO:53.

25. A standard, comprises a wild-type plasmid, a mutant plasmid, or both a wild-type plasmid and a mutant plasmid, said wild-type plasmid comprising a wild-type EGFR sequence, and said mutant plasmid comprising a mutant EGFR sequence.

26. The standard according to claim 25, wherein said wild-type EGFR sequence in said wild-type plasmid is SEQ ID NO:46, SEQ ID NO:48 or SEQ ID NO:52.

27. The standard according to claim 25, wherein said mutant plasmid comprises a mutation which is in a form of base G or A at position 2155 in EGFR Exon 18; a deletion from position 2235 to position 2249, from position 2236 to 2250, or from position 2254 to 2277 in Exon 19; or base T or G at position 2573 in Exon 21.

28. The standard according to claim 25, wherein said mutant EGFR sequence in said mutant plasmid is SEQ ID NO:47, SEQ ID NO:49, SEQ ID NO:50, SEQ ID NO:51 or SEQ ID NO:53.


SEQUENCE LISTING (53 sequences)

SEQ ID NO: 1 - DNA, 3633 bp, Unknown
  Sequence of EGFR Exon (ref. NCBI NM_005228; positions 2155, 2235-2277 and 2573 are underlined)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaagcttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 2 - DNA, 200 bp, Unknown
  a part of wild-type DNA sequence of EGFR 18 (ref. NCBI NT_033968.5)
gctgaggtgacccttgtctctgtgttcttgtcccccccagcttgtggagcctcttacacccagtggagaagctcccaaccaagctctcttgaggatcttgaaggaaactgaattcaaaaagatcaaagtgctgggctccggtgcgttcggcacggtgtataaggtaaggtccctggcacaggcctctgggctgggccgca

SEQ ID NO: 3 - DNA, 300 bp, Unknown
  a part of wild-type DNA sequence of EGFR 19 (ref. NCBI NT_033968.5)
cgtcacagcccccagcaatatcagccttaggtgcggctccacagccccagtgtccctcaccttcggggtgcatcgctggtaacatccacccagatcactgggcagcatgtggcaccatctcacaattgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgtgggggtccatggctctga

SEQ ID NO: 4 - DNA, 300 bp, Unknown
  a part of wild-type DNA sequence of EGFR 21 (ref. NCBI NT_033968.5)
ttcttcccatgatgatctgtccctcacagcagggtcttctctgtttcagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtaaggaggtggctttaggtcagccagcattttcctgacaccagggaccaggctgccttcccactagctgtattgtttaacacatgcaggggagg

SEQ ID NO: 5 - DNA, 20 bp, Artificial
  upstream primer of EGFR Exon 18
gaggatcttgaaggaaactg

SEQ ID NO: 6 - DNA, 19 bp, Artificial
  upstream primer of EGFR Exon 18
ccagcttgtggagcctctt

SEQ ID NO: 7 - DNA, 19 bp, Artificial
  downstream primer of EGFR Exon 18
gccagggaccttaccttat

SEQ ID NO: 8 - DNA, 21 bp, Artificial
  downstream primer of EGFR Exon 18
ctgtgccagggaccttacctt

SEQ ID NO: 9 - DNA, 19 bp, Artificial
  upstream primer of EGFR Exon 19
cccagaaggtgagaaagtt

SEQ ID NO: 10 - DNA, 20 bp, Artificial
  upstream primer of EGFR Exon 19
gggactctggatcccagaag

SEQ ID NO: 11 - DNA, 18 bp, Artificial
  downstream primer of EGFR Exon 19
cctgaggttcagagccat

SEQ ID NO: 12 - DNA, 19 bp, Artificial
  downstream primer of EGFR Exon 19
cccacacagcaaagcagaa

SEQ ID NO: 13 - DNA, 20 bp, Artificial
  upstream primer of EGFR Exon 21
gcagccaggaacgtactggt

SEQ ID NO: 14 - DNA, 20 bp, Artificial
  upstream primer of EGFR Exon 21
ccctcacagcagggtcttct

SEQ ID NO: 15 - DNA, 18 bp, Artificial
  downstream primer of EGFR Exon 21
gtgggaaggcagcctggt

SEQ ID NO: 16 - DNA, 18 bp, Artificial
  downstream primer of EGFR Exon 21
gtgggaaggcagcctggt

SEQ ID NO: 17 - DNA, 21 bp, Artificial
  upstream primer of the G->A mutation at EGFR Exon 18 position 2155
tgctgagctccggtgcgttcg

SEQ ID NO: 18 - DNA, 21 bp, Artificial
  downstream primer of the G->A mutation at EGFR Exon 18 position 2155
ggagctcagcactttgatctt

SEQ ID NO: 19 - DNA, 21 bp, Artificial
  upstream primer of the deletion at EGFR Exon 19 position 2235-2249
atcaaaacatctccgaaagcc

SEQ ID NO: 20 - DNA, 21 bp, Artificial
  downstream primer of the deletion at EGFR Exon 19 position 2235-2249
atgttttgatagcgacgggaa

SEQ ID NO: 21 - DNA, 21 bp, Artificial
  upstream primer of the deletion at EGFR Exon 19 position 2236-2250
tcaagacatctccgaaagcca

SEQ ID NO: 22 - DNA, 21 bp, Artificial
  downstream primer of the deletion at EGFR Exon 19 position 2236-2250
gatgtcttgatagcgacggga

SEQ ID NO: 23 - DNA, 21 bp, Artificial
  upstream primer of the deletion at EGFR Exon 19 position 2254-2277
caacactcgatgtgagtttct

SEQ ID NO: 24 - DNA, 21 bp, Artificial
  downstream primer of the deletion at EGFR Exon 19 position 2254-2277
tcgagtgttgcttctcttaat

SEQ ID NO: 25 - DNA, 21 bp, Artificial
  upstream primer of the T->G mutation at EGFR Exon 21 position 2573
tgggcgggccaaactgctggg

SEQ ID NO: 26 - DNA, 21 bp, Artificial
  downstream primer of the T->G mutation at EGFR Exon 21 position 2573
tggcccgcccaaaatctgtga

SEQ ID NO: 27 - DNA, 18 bp, Artificial
  wild-type probe of EGFR Exon 18
ggctccggtgcgttcggc

SEQ ID NO: 28 - DNA, 21 bp, Artificial
  wild-type probe of EGFR Exon 18
cggagcccagcactttgatct

SEQ ID NO: 29 - DNA, 19 bp, Artificial
  probe of the G->A mutation at EGFR Exon 18 position 2155
tgctgagctccggtgcgtt

SEQ ID NO: 30 - DNA, 22 bp, Artificial
  probe of the G->A mutation at EGFR Exon 18 position 2155
cggagctcagcactttgatctt

SEQ ID NO: 31 - DNA, 25 bp, Artificial
  wild-type probe of EGFR Exon 19
tcaaggaattaagagaagcaacatc

SEQ ID NO: 32 - DNA, 26 bp, Artificial
  wild-type probe of EGFR Exon 19
cggagatgttgcttctcttaattcct

SEQ ID NO: 33 - DNA, 21 bp, Artificial
  probe of the deletion at EGFR Exon 19 position 2235-2249
cccgtcgctatcaaaacatct

SEQ ID NO: 34 - DNA, 21 bp, Artificial
  probe of the deletion at EGFR Exon 19 position 2235-2249
agatgttttgatagcgacggg

SEQ ID NO: 35 - DNA, 22 bp, Artificial
  probe of the deletion at EGFR Exon 19 position 2236-2250
cccgtcgctatcaagacatctc

SEQ ID NO: 36 - DNA, 22 bp, Artificial
  probe of the deletion at EGFR Exon 19 position 2236-2250
gagatgtcttgatagcgacggg

SEQ ID NO: 37 - DNA, 23 bp, Artificial
  probe of the deletion at EGFR Exon 19 position 2254-2277
aattaagagaagcaacactcgat

SEQ ID NO: 38 - DNA, 23 bp, Artificial
  probe of the deletion at EGFR Exon 19 position 2254-2277
atcgagtgttgcttctcttaatt

SEQ ID NO: 39 - DNA, 20 bp, Artificial
  wild-type probe of EGFR Exon 21
tggccagcccaaaatctgtg

SEQ ID NO: 40 - DNA, 23 bp, Artificial
  wild-type probe of EGFR Exon 21
aagatcacagattttgggctggc

SEQ ID NO: 41 - DNA, 19 bp, Artificial
  probe of the T->G mutation at EGFR Exon 21 position 2573
tggcccgcccaaaatctgt

SEQ ID NO: 42 - DNA, 21 bp, Artificial
  probe of the T->G mutation at EGFR Exon 21 position 2573
gatcacagattttgggcgggc

SEQ ID NO: 43 - DNA, 18 bp, Artificial
  downstream primer of EGFR Exon 18
tgggatccagagtccctt

SEQ ID NO: 44 - DNA, 20 bp, Artificial
  downstream primer of EGFR Exon 19
gtctttgtgttcccggacat

SEQ ID NO: 45 - DNA, 20 bp, Artificial
  downstream primer of EGFR Exon 21
gcctccttctgcatggtatt

SEQ ID NO: 46 - DNA, 18 bp, Unknown
  nucleic acid sequence containing G at EGFR Exon 18 position 2155 (position 2155 is underlined)
aaagtgctgggctccggt

SEQ ID NO: 47 - DNA, 18 bp, Unknown
  nucleic acid sequence containing A at EGFR Exon 18 position 2155 (position 2155 is underlined)
aaagtgctgggctccggt

SEQ ID NO: 48 - DNA, 53 bp, Unknown
  nucleic acid sequence containing wild-type sequence at EGFR Exon 18 position 2235-2277 (position 2235-2277 is underlined)
tcaaggaattaagagaagcaacatctccgaaagccaacaaggaaatcctcgat

SEQ ID NO: 49 - DNA, 38 bp, Unknown
  nucleic acid sequence containing the deletion at EGFR Exon 19 position 2235-2249
cgctatcaaaacatctccgaaagccaacaaggaaatcc

SEQ ID NO: 50 - DNA, 39 bp, Unknown
  DNA sequence containing the deletion at EGFR Exon 19 position 2236-2250
cgtcgctatcaagacatctccgaaagccaacaaggaaat

SEQ ID NO: 51 - DNA, 35 bp, Unknown
  DNA sequence containing the deletion at EGFR Exon 19 position 2254-2277
aagagaagcaacactcgatgtgagtttctgctttg

SEQ ID NO: 52 - DNA, 18 bp, Unknown
  DNA sequence containing T at EGFR Exon 21 position 2573 (position 2573 is underlined)
acagattttgggctggcc

SEQ ID NO: 53 - DNA, 18 bp, Unknown
  DNA sequence containing G at EGFR Exon 21 position 2573 (position 2573 is underlined)
acagattttgggcgggcc